(19) United States
(12) Patent Application Publication
(10) Pub. No.: US 2024/0277014 A1
(43) Pub. Date: Aug. 22, 2024

(54) TEMPEH CHIPS WITH IMPROVED ACID VALUE STABILITY AND METHOD OF MANUFACTURING THE SAME

(71) Applicant: Sunah CHUN, Seoul (KR)

(72) Inventor: Sunah CHUN, Seoul (KR)

(21) Appl. No.: 18/465,328

(22) Filed: Sep. 12, 2023

(30) Foreign Application Priority Data
Feb. 21, 2023 (KR) 10-2023-0023174

(51) Int. Cl. A23L 11/50; A23L 5/10; A23L 11/70; C12N 1/14

(52) U.S. Cl. CPC A23L 11/50; A23L 5/11; A23L 5/13; A23L 11/70; C12N 1/14

(57) ABSTRACT

A method for manufacturing tempeh chips with improved acid value stability and rancidity resistance includes frying tempeh made with black-eyed beans in coconut oil. The tempeh chips produced by such a method exhibits a stable acid value, and maintain unique nutrients and taste while being able to meet the acid value standards and requirements for imports and exports.

### CROSS-REFERENCE TO RELATED APPLICATION(S)

This application claims priority from Korean Application No. 10-2023-0023174 filed on Feb. 21, 2023, which is incorporated herein by reference in its entirety.

### TECHNICAL FIELD

The present disclosure relates to a method of manufacturing tempeh chips, more particularly, to a method of manufacturing tempeh chips having a more stable acid value by using cowpea beans (black-eyed beans) and coconut oil.

### RELATED ART

Tempeh (or tempe) is traditional fermented soybean food originated from Indonesia. Tempeh is generally prepared by boiling and peeling soybeans, inoculating Rhizopus strains and fermenting them. Tempeh is fermented soybean food; it is nutritious food that contains abundant proteins, probiotics, dietary fiber, vitamins, and minerals. In addition, tempeh can be consumed in a variety of ways, such as being cut into small sizes and fried or stir-fried, and is an approachable vegan food. It has recently become an internationally trendy food, aligned with various health-related consumer trends.

However, most countries have strict acid value standards for imported food items such as fried snacks in order to ensure the safety of the imported food in their markets. Tempeh chips that use soybeans, which are manufactured by frying tempeh as a savory confectionery, often fail to meet the requirements for the acid value index of 2.0 mg KOH/g FFA. Therefore, importing and exporting these tempeh chips can be challenging.

The usage of soybeans as raw materials can cause acid value levels to be higher than what is required since the relatively high fat contents of the soybeans can be oxidated during the frying.

Therefore, it is necessary to develop a method of manufacturing tempeh chips with improved acid value stability, while retaining the nutritional benefits of tempeh.

### SUMMARY

An aspect of the present disclosure provides tempeh chips with improved stability of acid values, that can retain a stable acid value level below the acid value level standard required by most countries, and has an extended shelf-life.

Another aspect of the present disclosure provides a method for producing such tempeh chips with improved acid value stability with a formula that utilizes cowpeas (black-eyed beans) cooked in coconut oil.

According to the present disclosure, a method of producing tempeh chips may include frying beans in coconut oil. The following features may be included individually or in any combinations.

The frying may be performed at about 150 to about 180° C. The frying may be performed for about 5 to about 7 minutes. An acid value of the tempeh chips may be about 2.0 mg KOH/g FFA or less.

In some embodiments, before performing the frying, the method may further include soaking the beans in water at about 60 to about 70° C. for about 3 to about 10 minutes.

In some embodiments, the method may further include germinating the beans. For example, the germination can be performed for about 24 to about 30 hours. During the germination step, radicles may be developed to about 1 to about 2 mm. The method may further include removing husks of the beans.

The method may further include soaking the husk-removed beans in vinegar-added water for about 15 to about 17 hours. The method may further include boiling the soaked beans in water for about 5 to about 15 minutes. The method may further include cooling the boiled beans, mixing the beans with Rhizopus oligosporus, and storing the beans to prepare fermented tempeh.

In some embodiments, the storing may include a first storage step performed at about 29 to about 32° C. for about 20 to about 25 hours, and a second storage step performed at about 28 to about 30° C. for about 7 to about 12 hours. During the second storage step, an internal temperature of the tempeh may be maintained at about 46 to about 48° C.

The second storage step may include slicing the tempeh to a thickness of about 1.0 to about 1.5 mm. A moisture content of the sliced tempeh may be about 44% or more and a pH between about 4.5 and about 6.0.

In some embodiments, the beans may include black-eyed beans.

Another aspect of the present disclosure includes tempeh chips produced by the method described herein.

The manufacturing process of tempeh chips according to the present disclosure can preserve the nutritional benefits and the inherent savory taste of tempeh. The produced tempeh chips can have a lasting acid value stability that allows the products to be distributed for a longer period of time from date of manufacture.

The advantages of the present disclosure are not limited thereto, and other advantages can be understood from the following description.

### BRIEF DESCRIPTIONS OF THE DRAWINGS

FIG. 1 shows the tempeh mixture being fermented in a fermentation room. The tempeh mixture is placed into a plastic bag (or a tube), with its ends tied with a clip, and fermented at the temperatures of 50° C., 48° C., and 45° C.

FIG. 2 shows tempeh chips made with cowpeas (black-eyed beans) and coconut oil that were stored for 24 hours in a first fermentation chamber, and fermented for additional 8 hours in a second fermentation chamber.

### DETAILED DESCRIPTION

The present disclosure provides a method for producing tempeh chips with improved stability of acid values, by the process of cooking the black-eyed beans in coconut oil. By frying the black-eyed beans in coconut oil, the tempeh chips according to the present disclosure have an excellent acid value stability and rancidity resistance while maintaining the nutty taste and retaining its high nutritional values.

Cowpeas used in the present disclosure are best known in English as “black-eyed beans,” due to the small black spot at the center of each bean. Herein, the term “black-eyed beans” will be interchangeably used with cowpeas throughout the present disclosure.

Black-eyed beans are low in calories, rich in proteins and carbohydrates, high in iron and potassium, have a sweet flavor, and are easy to digest. These beans are often used as the ingredients in dietary-supplement food or health food that are popular among people of all ages. The origin of black-eyed beans is generally known as west Africa, but it has recently been cultivated worldwide-including the United States, India, and China. Cultivation of these beans is also possible in the climate of South Korea, during the accommodating climate from July to September, where production mainly occurs.

Because black-eyed beans have lower fat contents, the tempeh chips according to the present disclosure can benefit from the compositional traits in maintaining a stable acid value.

Coconut oil used in the present disclosure is a kind of vegetable oil, extracted by drying the flesh of coconut fruit and then pressing it without addition of any chemical substances. It is manufactured using coconuts harvested from Southeast Asia, India, South America, and Africa, where palm trees are frequently grown.

While most vegetable oils are composed of unsaturated fatty acids, coconut oil contains a large amount of saturated fatty acids, so it solidifies below room temperature.

The tempeh chips according to the present disclosure may maintain a stable acid value, and are savory and unique in taste, due to the usage of black-eyed beans and coconut oil.

In the present disclosure, the frying (e.g., deep-frying) of the prepared tempeh in the coconut oil may refer to a process of immersing the food in the oil and frying it.

The frying process according to the present disclosure may be performed while the coconut oil is maintained at a temperature ranging from about 130° C. to about 190° C. For example, the temperature of the coconut oil may be maintained at a temperature of about 140 to about 190° C., about 150 to about 190° C., about 150 to about 180° C., about 150 to about 170° C., about 150 to about 160° C., about 160 to about 190° C., about 170 to about 190° C., about 175 to about 190° C., about 180 to about 190° C., or about 185 to about 190° C.

In some embodiments, the process may be performed between about 150 and about 160° C. Due to the characteristics of tempeh, the frying may be performed at a low temperature because the high fat component contained in the beans, which are the raw material, may be easily burned.

In addition, frying time according to the present disclosure may be about 3 to about 15 minutes. For example, the frying time may be about 3 to about 10 minutes, about 3 to about 7 minutes, about 5 to about 15 minutes, about 5 to about 10 minutes, about 5 to about 7 minutes, about 7 to about 15 minutes, about 7 to about 10 minutes, or about 10 to about 15 minutes. These frying times give the tempeh chips an excellent, crispy texture and appetizing golden color.

As used herein, the term “acid value stability” may mean a state in which the oxidation of oil and/or beans in the production of tempeh is retarded or prevented, and in which a more stable and low acid value is maintained. Tempeh chips with improved acid value stability can eliminate or minimize the occurrence of rancidity, prevent rancid taste, and reduce health risks. Accordingly, tempeh chips with improved acid value stability can be distributed with longer shelf-lives from the date of manufacture, with a more stable acid value that is less than the maximum value required by the importing countries.

As used herein, the term “acid value” may be defined as the amount of potassium hydroxide (KOH) required to neutralize free fatty acid (FFA) in 1 g of the specimen, which is expressed in milligrams (i.e., mg of KOH per g of FFA). This means that the content of free fatty acids is indirectly measured as the number of mg of potassium hydroxide required to neutralize it. Tempeh chips with improved acid value stability according to the present disclosure may have an acid value of 2.0 mg KOH/g FFA or less. For example, the acid value may be maintained stably at about 0.7 mg KOH/g FFA during the transportation, distribution, and storage. Since all food can become oxidized over time in general, the acid value gradually increases as time elapses after the food is prepared.

Tempeh chips with improved acid value stability according to the present disclosure may additionally include an antioxidant. An antioxidant is a substance that inhibits oxidation. Widely used antioxidants include butylated hydroxytoluene (BHT) and tocopherol, but the present disclosure is not limited thereto. The present disclosure may use BHT, but may also use tocopherol.

In some embodiments, the method according to the present disclosure may include a step of soaking the black-eyed beans in water and removing the skins of the black-eyed beans before performing the step of frying the black-eyed beans in coconut oil. The step of soaking the black-eyed beans in water may be performed while the temperature of water is maintained at about 50 to about 80° C. For example, the soaking may be performed at about 50 to about 70° C., about 60 to about 80° C., or about 60 to about 70° C. Further, the soaking may be performed for about 2 to about 15 minutes. For example, the soaking may be performed for about 2 to about 13 minutes, about 2 to about 10 minutes, about 3 to about 15 minutes, about 3 to about 13 minutes, or about 3 to about 10 minutes. By soaking the black-eyed beans, they can be peeled more easily without clumping or breaking.

In some embodiments, the method according to the present disclosure may include a step of germinating the black-eyed beans. Without being bound by any particular theory, the germination process may increase the protein contents in the tempeh. Traditional soybean tempeh typically includes about 6 grams of protein in a 50-gram package. However, black-eyed bean tempeh may have lower protein contents than soybean tempeh due to the nature of the type of the beans. For example, black-eyed bean tempeh may include about 4 grams of protein in a 50-gram package. In order to increase the protein content, the black-eyed beans may be germinated before being made to tempeh.

By way of example, the black-eyed beans may be germinated for about 12 to about 48 hours. For example, the germination may be performed for about 18 to about 36 hours, for about 24 to about 36 hours, or for about 24 to about 30 hours. After the germination process, radicles may develop in the black-eyed beans. For example, after 24 to about 30 hours of germination, about 1 to about 2 mm of radicles may develop in the black-eyed beans. With the germination process added, the protein content of the black-eyed bean tempeh may increase to about 5.2 g in a 50-gram package.

The method according to the present disclosure may further include the step of soaking the peeled black-eyed beans in water to which vinegar is added. Soaking of the black-eyed beans in vinegar-added water may be performed for about 12 to about 20 hours, for example, about 12 to about 17 hours, about 15 to about 20 hours, or about 15 to about 17 hours. By soaking the peeled black-eyed beans in vinegar-added water, the fermentation may be facilitated and accelerated in making tempeh.

The method according to the present disclosure may also include the steps of boiling the black-eyed beans soaked in vinegar-added water in water after washing them. The boiling in water may be performed for about 3 to about 20 minutes, for example, about 3 to about 15 minutes, about 5 to about 20 minutes, or about 5 to about 15 minutes. By cooking the beans in boiling water, fermentation may proceed more efficiently due to the removal of unnecessary microorganisms and moisture remaining on the surface of the black-eyed beans.

Fermented tempeh may be prepared by cooling the boiled black-eyed beans, mixing them with bacterial strains, and storing them for a predetermined period of time. The bacterial strain that may be used for the fermentation may include Rhizopus, and more specifically, Rhizopus oligosporus.

In this step, starch may also be mixed together with the beans and the bacterial strain. Starch is a high-molecular carbohydrate primarily composed of many glucose units linked by glycosidic bonds. When starch is mixed with water, a thick paste formulation is produced, which functions as a thickener, hardener, or adhesive in food. The starch used in the present disclosure may be extracted from a storage root of a cassava plant, also known as tapioca starch.

The period of fermentation of the tempeh may include a first (e.g., initial) storage step and a second (e.g., secondary) storage step. The first storage step may store the tempeh for about 20 to about 25 hours at about 29 to about 32° C. For example, the first storage step may be performed for about 24 hours at about 29 to about 32° C. The second storage step may be performed subsequent to the first storage step, and may be performed at about 28 to about 30° C. for about 6 to about 12 hours. For example, the second storage step may be performed for about 6 to about 12 hours, about 6 to about 10 hours, about 6 to about 8 hours, about 7 to about 12 hours, about 7 to about 10 hours, or about 7 to about 8 hours.

In the storage steps of the present disclosure, the first storage step and the second storage step may be combined to be within about 29 to about 32 hours. If the storage step exceeds about 32 hours, the tempeh may be over-fermented, and unpleasant odors may be generated, reducing its commercial value.

The method according to the present disclosure may further include the step of cutting (e.g., slicing) the tempeh to a thickness of about 1.0 to about 1.5 mm. The cutting step may be performed during the second storage step. The thickness of the tempeh is not limited to the above-mentioned range, and any thickness that is suitable and/or convenient for intake and optimized texture may be used.

In some embodiments, the slicing step may be performed during the second storage step, before 32 hours have elapsed. If slicing is performed after 32 hours have elapsed, acidity may increase in the tempeh.

The moisture content of the sliced tempeh may be at about 44% or higher, and the pH may be between about 4.5 and about 6.0. Since even a slight difference in the moisture content and/or pH may cause a noticeable difference in the taste of the tempeh chips, it is important to prepare tempeh that satisfies the above-mentioned range.

The advanced recipe according to the present disclosure may produce tempeh chips with improved stability for acid values and may exhibit improved rancidity resistance through the above-described manufacturing process. Tempeh chips manufactured through the above-described process may preserve the flavor and nutritional components of tempeh chips, and may also retain improved rancidity resistance, have a stable acid value below the standard values required by the importing countries, and accordingly, they can be distributed for a longer period of time from the date of manufacture.

Hereinafter, the examples for manufacturing tempeh chips according to the present disclosure will be described in detail.

### Manufacturing Example 1: Manufacturing Method of Tempeh Chips

### Preparation Example 1.1: Preparation of the Black-Eyed Beans

In this preparation example, 1 kg of fresh black-eyed beans were prepared. The black-eyed beans should have dark brown skin, be free of wrinkles and pest contamination. The skins (e.g., husks) of the black-eyed beans were removed prior to boiling. However, removing the skins may be challenging due to the small size of the black-eyed beans, and therefore, in order to more easily and efficiently remove the skins, the black-eyed beans were soaked in water with a temperature of about 60 to about 70° C. for about 5 minutes. After the soaking, the skins of the black-eyed beans were removed using a machine.

The skinned black-eyed beans were boiled in water for about 40 minutes. By boiling after removing the skin, the black-eyed beans may keep its round shape without being crushed or squashed. After the black-eyed beans were boiled, the black-eyed beans were rinsed with cold, running water at a temperature of about 10 to about 20° C.

Subsequently, a 3% equivalent of vinegar-added water was prepared by adding vinegar in about 500 g of water. The rinsed beans were soaked in the vinegar-water solution for about 16 hours. Subsequently, the soaked black-eyed beans were washed again with cold water.

The washed black-eyed beans were boiled again in water for about 10 minutes. By boiling the black-eyed beans, moisture and microorganisms remaining in the beans may be removed, and the fermentation process may be facilitated more efficiently.

The boiled black-eyed beans were transferred to a tray that is lined with a filter cloth to remove excess water, and cooled with a fan. During the cooling, the beans should be carefully monitored so as not to inactivate or kill the strain of Rhizopus oligosporus required for fermentation.

### Preparation Example 1.2: Preparation of Tempeh Mixture

2 g of Rhizopus oligosporus strain, and 1000 g of tapioca starch were prepared and mixed in to 1.5 kg of the cooled black-eyed beans. The strains were mixed first, and when they were sufficiently mixed, the tapioca starch was added to prepare the final mixture. Adding the tapioca starch may give the tempeh chips its crispy texture.

Before starting the fermentation of the mixture, the moisture content of the mixture was checked and confirmed that it was at least 46%. Suitable moisture content for tempeh is 46% or above in order for fermentation to properly occur, so that the tempeh does not dry out and become crumbly when sliced.

The mixture was placed in plastic bags, one or both ends of which were open, for fermentation. The ends of the plastic bags were tied and secured using clips, and the bagged mixture was stored for 24 hours in the first fermentation room at a temperature of about 29 to about 31° C. and a humidity of about 60 to about 70% (the first storage step).

After the initial 24 hours in the first fermentation chamber, further fermentation was performed in the second fermentation chamber for 6 to 8 hours. The temperature of the fermentation chamber was about 28 to about 30° C., which was adjusted based on the internal temperature of the tempeh mixture (second storage step).

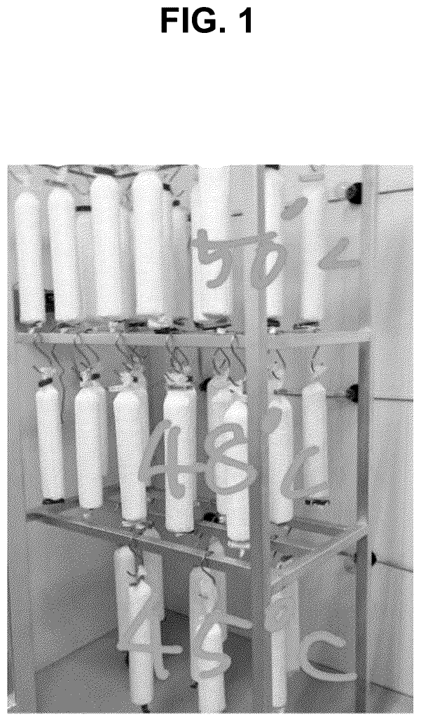

If the internal temperature of the tempeh is higher than 48° C., the tempeh may become too acidic (e.g., due to over-fermentation), and if the internal temperature is lower than 46° C., the tempeh may break or crumble when sliced. The conditions were adjusted accordingly (FIG. 1).

While fermenting the tempeh during the second storage step, it was sliced to a thickness of about 1.0 to about 1.5 mm using a slicing machine. If the tempeh is cut during the first storage step, it can be easily broken and it is more difficult to maintain its circular shape, and if it is cut after 32 hours of the second storage step, the tempeh can become over-fermented.

After the tempeh is cut, the moisture content is required to be at least 44% and have a pH of 4.5 to 6.0. Since the degree of success of fermentation may vary even with slight differences in the moisture and pH, it is important to maintain the above ranges in order to consistently maintain the taste of tempeh chips.

### Preparation Example 1.3: Manufacturing of Tempeh Chips

6 L of coconut oil (such as the one that is commercially available from Barco Co.) was prepared with 1.08 g of BHT added as the antioxidant. When BHT is added as an antioxidant, rancidity of fat may be prevented. In addition, a solution of 15 g of salt, 30 g of minced garlic, and 1500 L of water was prepared for dipping.

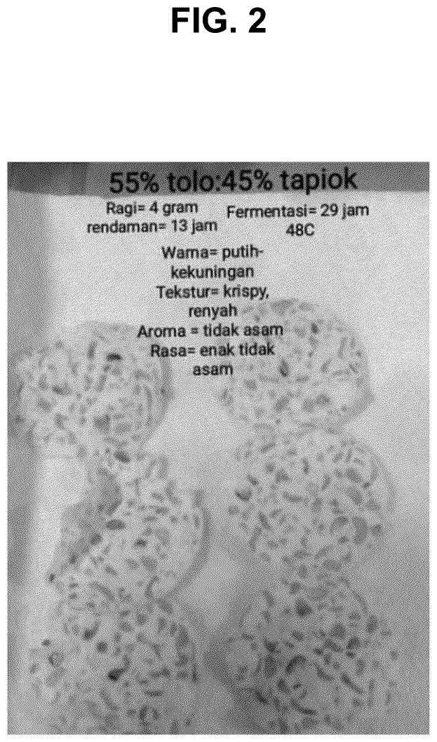

The sliced tempeh was immersed in the dipping water and then removed. Subsequently, it was fried using the coconut oil at a temperature of about 150° C. for about 5 to about 7 minutes until it became crisp, and then cooled (FIG. 2).

After the tempeh chips were cooled, the moisture content of the chips was checked to confirm that it did not exceed 3%.

Then, seasoning powder was added to the tempeh chips and stirred to mix well. The moisture content of the chips was checked to confirmed that it did not exceed 2%. Tempeh chips were then packaged in an aluminum packaging material, along with oxygen absorbers (e.g., Oxymove®) and nitrogen.

### Example 1. Acid Value Test of Tempeh Chips

### Example 1.1. Acid Value Test of Tempeh Chips Prepared Using Soybean and Coconut Oil

Tempeh chips were prepared similarly to the method according the present disclosure as described above, except that the tempeh chips were prepared by frying soybeans in coconut oil. The acid value was measured across the variety of seasoning prior to packaging.

TABLE 1

| Frying | Acid Value | |
|---|---|---|
| Conditions | (mg KOH/g FFA) | Seasoning |
| Tempeh made | 7.11 | BBQ |
| with soybeans | 7.10 | No Seasoning |
| is fried in | 7.60 | Cheese |
| coconut oil | 6.53 | Original Seasoning |
| at 150° C. | 7.20 | Hot Chili |

As shown in Table 1 above, all of the tempeh chips prepared using the combination of soybeans and coconut oil recorded high acid values regardless of the type of seasoning. Even when no seasoning was treated, a high acid value of 7.10 mg KOH/g FFA was recorded after frying.

### Example 1.2. Acid Value Test of Tempeh Chips Prepared Using Soybeans and Various Types of Oils

Tempeh chips were prepared by frying soybeans in coconut oil, palm oil, sunflower oil, and rice bran oil, and the acid value of each was measured.

TABLE 2

| Frying oil and | | | | Acid Value |
|---|---|---|---|---|
| antioxidant | Frying | | FFA | (mg KOH/g |
| combination | time | | (%) | FFA) |
| Coconut oil 75%, | 0 | minutes | 1.20 | 6.29 |
| Palm oil 25%, | 20 | minutes | 1.78 | |
| Tocopherol 0.36 g/L | 1 | hour | 0.79 | |
| Coconut oil 50%, | 0 | minutes | 0.57 | 7.16 |
| Palm oil 50%, | 20 | minutes | 0.39 | |
| Tocopherol 0.36 g/L | 1 | hour | 2.68 | |
| Coconut oil 25%, | 0 | minutes | 0.19 | 6.91 |
| Palm oil 75%, | 20 | minutes | 0.19 | |
| Tocopherol 0.36 g/L | 1 | hour | 1.55 | |
| Coconut oil 100%, | 0 | minutes | 1.00 | — |
| Tocopherol 0.36 g/L | 20 | minutes | 0.49 | |
| | 1 | hour | 0.34 | |
| Palm oil 100% (1 L), | 0 | minutes | 0.28 | — |
| BHT 0.36 g | 20 | minutes | 0.96 | 5.20 |
| | 1 | hour | 1.18 | 7.30 |
| Palm oil 100% (1 L), | 0 | minutes | 0.84 | 6.91 |
| Tocopherol 0.36 g | 20 | minutes | 1.32 | |
| | 1 | hour | 1.95 | |
| Sunflower oil 100% (1 L), | 1 | hour | — | 5.70 |
| Tocopherol 0.36 g | 2 | hours | — | |
| | 3 | hours | — | |
| Rice bran oil 100% (1 L) | 2 | hours | — | 4.70 |

As shown in the table above, the tempeh chips using soybeans showed a high acid value across all other types of oils, with no significant effect depending on the type of fat or antioxidant, or the blending ratio of the oil. On the other hand, in the case of frying in 100% coconut oil, the FFA content was recorded lower than for the mixture of the coconut and palm oils. Coconut oil was thus shown to help maintaining a stable acid value.

### Example 1.3. Acid Value Test of Tempeh Chips Prepared Using Black-Eyed Beans and Coconut Oil

4000 g of black-eyed beans, 3,272 g of tapioca starch and 2-3 g of Rhizopus oligosporus were combined and prepared according to the method described in Preparation Example 1 above.

The soaking of the black-eyed beans was carried out for 11 hours, and the fermentation was carried out for 31 hours.

First, the coconut oil was heated for 3 hours using 6 L of coconut oil and 2.16 g of tocopherol. In this example, as shown in Table 3 below, a stable FFA content range was confirmed.

TABLE 3

| | | |
|---|---|---|
| Frying oil content | Frying time | FFA (%) |

| | | | |
|---|---|---|---|
| Coconut oil 6 L and | 0 | minutes | 0.0358 |
| Tocopherol 2.16 g | 30 | minutes | 0.044 |
| | 1 | hour | 0.048 |
| | 1.5 | hours | 0.0491 |
| | 2 | hours | 0.0516 |
| | 2.5 | hours | 0.0572 |
| | 3 | hours | 0.0582 |

As indicated above, an attempt was made to test whether a stable acid value would be maintained even when frying in the coconut oil was performed over a longer time, using 6 L of coconut oil and 2.16 g of tocopherol.

As shown in Table 4, when frying was performed for 6 hours, the contents of FFA increased. After the 6 hour frying, 2 L of coconut oil and 0.72 g of tocopherol were added, and frying was performed for additional 1 hour, resulting in a total of 7 hours of frying. The acid value derived in this example was stably maintained at 1.31 mg KOH/g FFA.

TABLE 4

| Frying | Frying | | Acid Value |
|---|---|---|---|
| oil | time | FFA (%) | (mg KOH/g FFA) |
| Coconut oil 6 L and | 3 hours | 0.0461 | — |
| Tocopherol 2.16 g | 3.5 hours | 0.0486 | — |
| | 4 hours | 0.0513 | — |
| | 5 hours | 0.0553 | — |
| | 6 hours | 0.0759 | — |
| Additional Coconut | 7 hours | 0.0493 | 1.31 mg KOH/g FFA |
| Oil 2 L and | | | |
| Tocopherol 0.72 g | | | |

Even when the tempeh chips made using black-eyed beans were fried in coconut oil for longer than 7 hours, a low and stable acid value was exhibited.

As can be seen in Table 2 above, when the soybeans were fried in coconut oil, palm oil, sunflower oil, and rice bran oil, and when the acid values were measured after 1 to 3 hours, high acid values of 4.70 to 7.30 mg KOH/g FFA were recorded. In contrast, as can be seen in Table 4 above, when black-eyed beans were fried in coconut oil, low and stable acid values of 1.30 mg KOH/g FFA were recorded even after 6 to 7 hours.

The antioxidant efficacy of tocopherol compared to BHT were also evaluated by measuring the FFA content. As shown in Table 5 below, when frying for 2 hours, the contents of FFA increased. After the 2 hour period, 1.5 L of coconut oil and 0.4 g of BHT were further added. Coconut oil and BHT were additionally added and FFA was measured when 4.5 hours had passed since the initial frying process. With this example, it was confirmed that the acid value stability was better when BHT was used as an antioxidant, as the FFA values were lower than using tocopherol as the antioxidant.

TABLE 5

| | | |
|---|---|---|
| Frying oil | Frying time | FFA (%) |

| | | | |
|---|---|---|---|
| Coconut Oil 6 L and | 0 | minutes | 0.0463 |
| BHT 0.18 g/L | 30 | minutes | 0.0507 |
| | 1 | hour | 0.0594 |
| | 1.5 | hours | 0.0650 |
| | 2 | hours | 0.0801 |
| Coconut Oil 1.5 L and | 4.5 | hours | 0.0270 |
| BHT 0.4 g added | | | |

### Example 2. Sensory Assessment of Black-Eyed Beans Tempeh Chips Fried in Coconut Oil

As shown in Table 6, the tempeh chips prepared according to the present disclosure did not taste rancid, and they provided a crispy texture, resulting in tempeh chips with more desirable characteristics.

TABLE 6

| | Black-eyed beans 55% and | Black-eyed beans 60% and |
|---|---|---|
| | Tapioca Starch 45% | Tapioca Starch 40% |
| Color | White or golden yellow | Brown |

| | |
|---|---|
| Taste and smell | No trace of rancidity. Nutty and savory |
| Texture | Crunchy, crisp texture |

What is claimed is:

1. A method of producing tempeh chips, the method comprising:
frying beans in coconut oil.

2. The method of claim 1, wherein the frying is performed at about 150 to about 180° C.

3. The method of claim 1, wherein the frying is performed for about 5 to about 7 minutes.

4. The method of claim 1, wherein an acid value of the tempeh chips is about 2.0 mg KOH/g FFA or less.

5. The method of claim 1, further comprising, before performing the frying:
soaking the beans in water at about 60 to about 70° C. for about 3 to about 10 minutes.

6. The method of claim 5, further comprising:
germinating the beans for about 24 to about 30 hours.

7. The method of claim 6, further comprising:
removing husks of the beans.

8. The method of claim 7, further comprising:
soaking the husk-removed beans in vinegar-added water for about 15 to about 17 hours.

9. The method of claim 8, further comprising:
boiling the soaked beans in water for about 5 to about 15 minutes.

10. The method of claim 9, further comprising:
cooling the boiled beans;
mixing the beans with Rhizopus oligosporus; and
storing the beans to prepare fermented tempeh.

11. The method of claim 10, wherein the storing comprises:
a first storage step performed at about 29 to about 32° C. for about 20 to about 25 hours; and
a second storage step performed at about 28 to about 30° C. for about 7 to about 12 hours.

12. The method of claim 11, wherein, during the second storage step, an internal temperature of the tempeh is maintained at about 46 to about 48° C.

13. The method of claim 11, wherein the second storage step comprises:
slicing the tempeh to a thickness of about 1.0 to about 1.5 mm.

14. The method of claim 13, wherein a moisture content of the sliced tempeh is about 44% or more and a pH between about 4.5 and about 6.0.

15. The method of claim 1, wherein the beans include black-eyed beans.

16. Tempeh chips produced by the method of claim 1.
